(19) United States
(12) Patent Application Publication
(10) Pub. No.: US 2008/0208069 A1
(43) Pub. Date: Aug. 28, 2008

(54) System and methods of hierarchical cardiac event detection

(76) Inventors: Michael Sasha John, Larchmont, NY (US); Bruce Hopenfeld, Oceanport, NJ (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/710,904

(22) Filed: Feb. 27, 2007

(51) Int. Cl. A61B 5/0402

(52) U.S. Cl. 600/509

(57) ABSTRACT

A system for the detection of cardiac events occurring in a human patient is provided. At least two electrodes are included in the system for obtaining an electrical signal from a patient's heart. An electrical signal processor is electrically coupled to the electrodes for processing the electrical signal. An electrogram analysis scheme is described, according to which electrogram segments or individual beats are classified according to various features, and different cardiac event tests are applied based on this classification.

### FIELD OF USE

This invention is in the field of medical device systems that monitor a patient's cardiovascular condition.

### BACKGROUND OF THE INVENTION

Heart disease is the leading cause of death in the United States. A heart attack, also known as an acute myocardial infarction (AMI), typically results from a blood clot or “thrombus” that obstructs blood flow in one or more coronary arteries. AMI is a common and life-threatening complication of coronary artery disease. Coronary ischemia is caused by an insufficiency of oxygen to the heart muscle. Ischemia is typically provoked by physical activity or other causes of increased heart rate when one or more of the coronary arteries is narrowed by atherosclerosis. AMI, which is typically the result of a completely blocked coronary artery, is the most extreme form of ischemia. Patients will often (but not always) become aware of chest discomfort, known as “angina”, when the heart muscle is experiencing ischemia. Those with coronary atherosclerosis are at higher risk for AMI if the plaque becomes further obstructed by thrombus.

Acute myocardial infarction and ischemia may be detected from a patient's electrocardiogram (ECG) by noting an ST segment shift (i.e., voltage change). However, without knowing the patient's normal ECG pattern, detection from a standard 12 lead ECG can be unreliable.

Fischell et al. in U.S. Pat. Nos. 6,112,116, 6,272,379 and 6,609,023 describe implantable systems and algorithms for detecting the onset of acute myocardial infarction and providing both patient alerting and treatment. The Fischell et al. patents describe how the electrical signal from inside the heart can be used to determine various states of myocardial ischemia. In U.S. Pat. No. 6,609,023, Fischell et al. disclose a method for detecting a cardiac event based on both the ST segment and the T wave. The term “medical practitioner” shall be used herein to mean any person who might be involved in the medical treatment of a patient. Such a medical practitioner includes, but is not limited to, a medical doctor (e.g., a general practice physician, an internist or a cardiologist), a medical technician, a paramedic, a nurse or an electrogram analyst. Although the masculine pronouns “he” and “his” are used herein, it should be understood that the patient, physician or medical practitioner could be a man or a woman. A “cardiac event” includes an acute myocardial infarction, ischemia caused by effort (such as exercise) and/or an elevated heart rate, bradycardia, tachycardia or an arrhythmia such as atrial fibrillation, atrial flutter, ventricular fibrillation, and premature ventricular or atrial contractions (PVCs or PACs respectively).

It is generally understood that the term “electrocardiogram” is defined as the heart's electrical signals sensed by means of skin surface electrodes that are placed in a position to indicate the heart's electrical activity (depolarization and repolarization). An electrocardiogram segment refers to a portion of electrocardiogram signal that extends for either a specific length of time, such as 10 seconds, or a specific number of heart beats, such as 10 beats. A beat is defined as a sub-segment of an electrogram or electrocardiogram segment containing exactly one R wave. As used herein, the PQ segment of a patient's electrocardiogram or electrogram is the typically straight segment of a beat of an electrocardiogram or electrogram that occurs just before the R wave and the ST segment is a typically straight segment that occurs just after the R wave. As defined herein, the term “electrogram” is the heart's electrical signal voltage as sensed from one or more electrode(s) that are placed in a position, whether inside the body, on the body surface or off the body, to indicate the heart's electrical activity (depolarization and repolarization). An electrogram segment refers to a portion of the electrogram signal for either a specific length of time, such as 10 seconds, or a specific number of heart beats, such as 10 beats. For the purposes of this specification, the terms “detection” and “identification” of a cardiac event have the same meaning.

### SUMMARY OF THE INVENTION

The present invention includes electrodes placed to advantageously sense electrical signals from a patient's heart, resulting in an electrogram. According to the preferred embodiment, the electrogram is analyzed to detect myocardial ischemia. This is accomplished by hierarchically classifying the electrogram based on various characteristics, such as T wave amplitude and the polarity of an ST shift. An appropriate ischemia test is selected based on the classification. Ischemia tests preferably involve examining the sum of the ST/T segment, QRS duration/slope changes, and the duration of the ST segment and T wave. For example, depending on waveform classification, ischemia may be detected based on whether the sum of the ST/T segment is small or large. Additional test factors include the rate at which a waveform shape is changing.

### BRIEF DESCRIPTION OF THE DRAWINGS

FIG. 1 illustrates a Guardian system for the detection of a cardiac event and for warning the patient that a medically relevant cardiac event is occurring;

FIG. 2 is a block diagram of an implanted cardiosaver system;

FIGS. 3a-3c show various electrogram waveforms and their relationship to possible transmembrane potentials within the heart.

FIG. 4 shows examples of different types of QRS complexes and how DC offsets (e.g. TQ and ST voltages) relate thereto. Bruce, just a note: there is no T component in the figure.

FIG. 5 is a flowchart of the hierarchical electrogram waveform analysis that may be used to detect a cardiac condition.

FIG. 6 shows T wave and ST segment amplitudes as a function of heart rate.

FIG. 7 shows a possible implementation of a spline-based method for comparing electrogram shapes.

FIG. 8 shows a table that shows associations between parameter value ranges and a cardiac event such as ischemia.

### DETAILED DESCRIPTION OF THE INVENTION

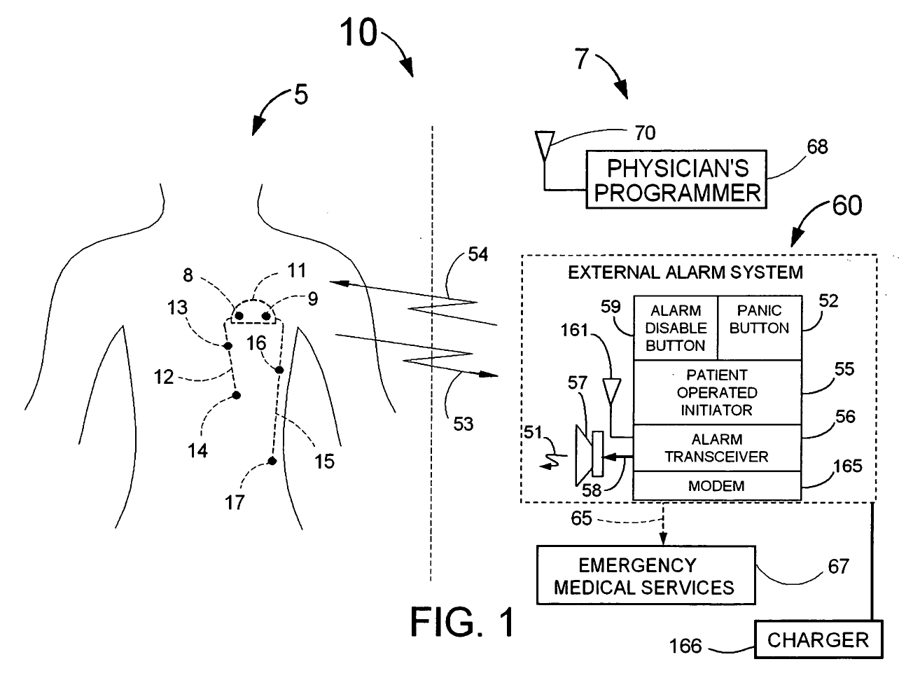

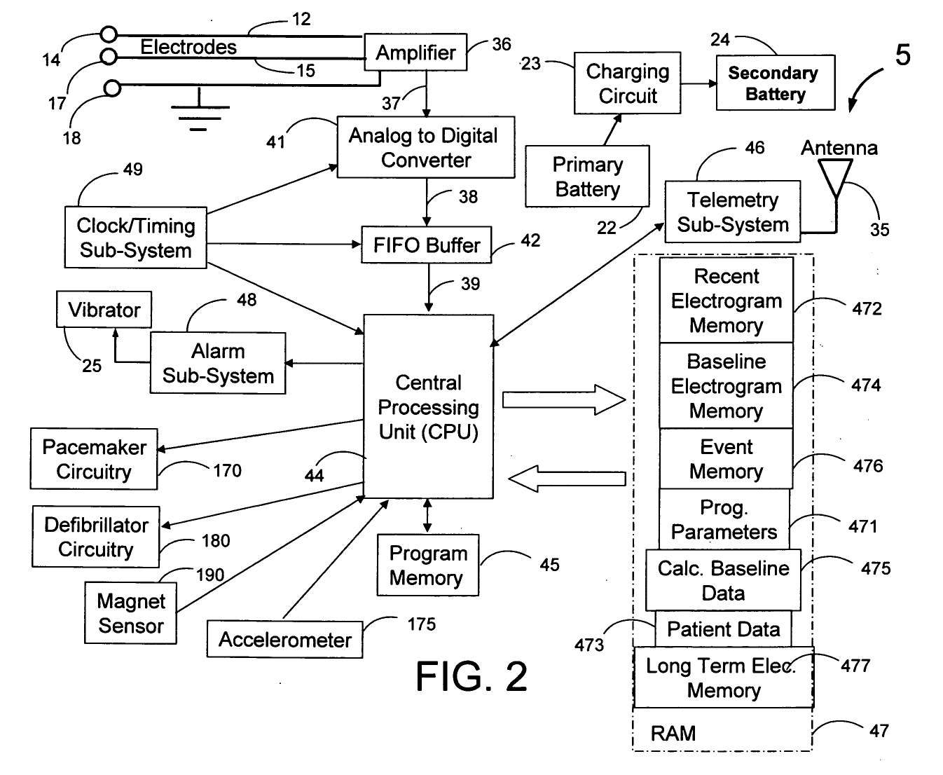

FIG. 1 illustrates one embodiment of the Guardian system 10 consisting of an implanted Cardiosaver 5 and external equipment 7. The battery powered Cardiosaver 5 contains electronic circuitry that can detect a cardiac event such as an acute myocardial infarction or arrhythmia and warn the patient when the event, or a clinically relevant precursor, occurs (Bruce, do we wait till the AMI occurs or are we trying to anticipate this you have defined AMI as heart attack above rather than simply ischemia.). The Cardiosaver 5 can store the patient's electrogram for later readout and can send wireless signals 53 to and receive wireless signals 54 from the external equipment 7. The functioning of the Cardiosaver 5 will be explained in greater detail with the assistance of FIG. 2.

The Cardiosaver 5 has two leads 12 and 15 that have multi-wire electrical conductors with surrounding insulation. The lead 12 is shown with two electrodes 13 and 14. The lead 15 has subcutaneous electrodes 16 and 17. In fact, the cardiosaver 5 could utilize as few as one lead or as many as three and each lead could have as few as one electrode or as many as eight electrodes. Furthermore, electrodes 8 and 9 could be placed on the outer surface of the Cardiosaver 5 without any wires being placed externally to the cardiosaver 5.

The lead 12 in FIG. 1 could advantageously be placed through the patient's vascular system with the electrode 14 being placed into the apex of the right ventricle. The lead 12 with electrode 13 could be placed in the right ventricle or right atrium or the superior vena cava similar to the placement of leads for pacemakers and Implantable Coronary Defibrillators (ICDs). The metal case 11 of the cardiosaver 5 could serve as another electrode. It is also conceived that the electrodes 13 and 14 could be used as bipolar electrodes. Alternately, the lead 12 in FIG. 1 could advantageously be placed through the patient's vascular system with the electrode 14 being placed into the apex of the left ventricle. The electrode 13 could be placed in the left atrium.

The lead 15 could advantageously be placed subcutaneously at any location where the electrodes 16 and/or 17 would provide a good electrogram signal indicative of the electrical activity of the heart. Again for this lead 15, the case 11 of the cardiosaver 5 could be an indifferent electrode and the electrodes 16 and/or 17 could be active electrodes or electrodes 16 and 17 could function together as bipolar electrodes. The cardiosaver 5 could operate with only one lead and as few as one active electrode with the case of the cardiosaver 5 being an indifferent electrode. The guardian system 10 described herein can readily operate with only two electrodes.

One embodiment of the cardiosaver device 5 using subcutaneous lead 15 would have the electrode 17 located under the skin on the patient's left side. This could be best located between 2 and 20 inches below the patient's left arm pit. The cardiosaver case 11 could act as the indifferent electrode and would typically be implanted under the skin on the left side of the patient's chest.

FIG. 1 also shows the external equipment 7 that consists of a physician's programmer 68 having an antenna 70, an external alarm system 60 including a charger 166. The external equipment 7 provides means to interact with the cardiosaver 5. These interactions include programming the cardiosaver 5, retrieving data collected by the cardiosaver 5 and handling alarms generated by the cardiosaver 5.

The purpose of the physician's programmer 68 shown in FIG. 1 is to set and/or change the operating parameters of the implantable cardiosaver 5 and to read out data stored in the memory of the cardiosaver 5 such as stored electrogram segments. This would be accomplished by transmission of a wireless signal 54 from the programmer 68 to the cardiosaver 5 and receiving of telemetry by the wireless signal 53 from the cardiosaver 5 to the programmer 68. When a laptop computer is used as the physician's programmer 68, it would require connection to a wireless transceiver for communicating with the cardiosaver 5. Such a transceiver could be connected via a standard interface such as a USB, serial or parallel port or it could be inserted into the laptop's PCMCIA card slot. The screen on the laptop would be used to provide guidance to the physician in communicating with the cardiosaver 5. Also, the screen could be used to display both real time and stored electrograms that are read out from the cardiosaver 5.

In FIG. 1, the external alarm system 60 has a patient operated initiator 55, an alarm disable button 59, a panic button 52, an alarm transceiver 56, an alarm speaker (transducer?) 57 and an antenna 161 and can communicate with emergency medical services 67 with the modem 165 via the communication link 65. Other components such as alarm transducers for different modalities (e.g. visual) and a microphone for verbal communication may also be included.

If a cardiac event is detected by the cardiosaver 5, an alarm message is sent by a wireless signal 53 to the alarm transceiver 56 via the antenna 161. When the alarm is received by the alarm transceiver 56 a signal 58 is sent to the loudspeaker 57. The signal 58 will cause the loudspeaker to emit an external alarm signal 51 to warn the patient that an event has occurred. Examples of external alarm signals 51 include a periodic buzzing, a sequence of tones and/or a speech message that instructs the patient as to what actions should be taken. Furthermore, the alarm transceiver 56 can, depending upon the nature of the signal 53, send an outgoing signal over the link 65 to contact emergency medical services 67. When the detection of an acute myocardial infarction is the cause of the alarm, the alarm transceiver 56 could automatically notify emergency medical services 67 that a heart attack has occurred and an ambulance could be sent to treat the patient and to bring him to a hospital emergency room.

If the remote communication with emergency medical services 67 is enabled and a cardiac event alarm is sent within the signal 53, the modem 165 will establish the data communications link 65 over which a message will be transmitted to the emergency medical services 67. The message sent over the link 65 may include any or all of the following information: (1) a specific patient is having an acute myocardial infarction or other cardiac event, (2) the patient's name, address and a brief medical history, (3) a map and/or directions to where the patient is located, (4) the patient's stored electrogram including baseline electrogram data and the specific electrogram segment that generated the alarm (5) continuous real time electrogram data, and (6) a prescription written by the patient's personal physician as to the type and amount of drug to be administered to the patient in the event of a heart attack. If the emergency medical services 67 includes an emergency room at a hospital, information can be transmitted that the patient has had a cardiac event and should be on his way to the emergency room. In this manner the medical practitioners at the emergency room could be prepared for the patient's arrival.

The communications link 65 can be either a wired or wireless telephone connection that allows the alarm transceiver 56 to call out to emergency medical services 67. The typical external alarm system 60 might be built into a Pocket PC or Palm Pilot PDA where the alarm transceiver 56 and modem 165 are built into insertable cards having a standardized interface such as compact flash cards, PCMCIA cards, multimedia, memory stick or secure digital (SD) cards. The modem 165 can be a wireless modem such as the Sierra AirCard 300 or the modem 165 may be a wired modem that connects to a standard telephone line. The modem 165 can also be integrated into the alarm transceiver 56.

The purpose of the patient operated initiator 55 is to give the patient the capability for initiating transmission of the most recently captured electrogram segment from the cardiosaver 5 to the external alarm system 60. This will enable the electrogram segment to be displayed for a medical practitioner.

Once an internal and/or external alarm signal has been initiated, depressing the alarm disable button 59 will acknowledge the patient's awareness of the alarm and turn off the internal alarm signal generated within the cardiosaver 5 and/or the external alarm signal 51 played through the speaker 57. If the alarm disable button 59 is not used by the patient to indicate acknowledgement of awareness of a SEE DOCTOR alert or an EMERGENCY alarm, it is envisioned that the internal and/or external alarm signals would stop after a first time period (an initial alarm-on period) that would be programmable through the programmer 68.

For EMERGENCY alarms, to help prevent a patient ignoring or sleeping through the alarm signals generated during the initial alarm-on period, a reminder alarm signal might be turned on periodically during a follow-on periodic reminder time period. This periodic reminder time is typically much longer than the initial alarm-on period. The periodic reminder time period would typically be 3 to 5 hours because after 3 to 5 hours the patient's advantage in being alerted to seek medical attention for a severe cardiac event like an AMI is mostly lost. It is also envisioned that the periodic reminder time period could also be programmable through the programmer 68 to be as short as 5 minutes or even continue indefinitely until the patient acknowledges the alarm signal with the button 59 or the programmer 68 is used to interact with the cardiosaver 5.

Following the initial alarm on-period there would be an alarm off-period followed by a reminder alarm on-period followed by an alarm off-period followed by another reminder alarm on-period and so on periodically repeating until the end of the periodic reminder time period.

The alarm off-period time interval between the periodic reminders might also increase over the reminder alarm on-period. For example, the initial alarm-on period might be 5 minutes and for the first hour following the initial alarm-on period, a reminder signal might be activated for 30 seconds every 5 minutes. For the second hour the reminder alarm signal might be activated for 20 seconds every 10 minutes and for the remaining hours of the periodic reminder on-period the reminder alarm signal might be activated for 30 seconds every 15 minutes.

The patient might press the panic button 52 in the event that the patient feels that he is experiencing a cardiac event. The panic button 52 will initiate the transmission from the cardiosaver 5 to the external alarm system 60 via the wireless signal 53 of both recent and baseline electrogram segments. The external alarm system 60 will then retransmit these data via the link 65 to emergency medical services 67 where a medical practitioner will view the electrogram data. The remote medical practitioner could then analyze the electrogram data and call the patient back to offer advice as to whether this is an emergency situation or the situation could be routinely handled by the patient's personal physician at some later time.

It is envisioned that there may be preset limits within the external alarm system 60 that prevent the patient operated initiator 55 and/or panic button from being used more than a certain number of times a day to prevent the patient from running down the batteries in the cardiosaver 5 and external alarm system 60 as wireless transmission takes a relatively large amount of power as compared with other functional operation of these devices.

The alarm signal associated with an excessive ST shift caused by an acute myocardial infarction can be quite different from the “SEE DOCTOR” alarm means associated with progressing ischemia during exercise. For example, the SEE DOCTOR alert signal might be an audio signal that occurs once every 5 to 10 seconds. A different alarm signal, for example an audio signal that is three buzzes every 3 to 5 seconds, may be used to indicate a major cardiac event such as an acute myocardial infarction. Similar alarm signal timing would typically be used for both internal alarm signals generated by the alarm sub-system 48 of FIG. 2 and external alarm signals generated by the external alarm system 60.

In any case, a patient can be taught to recognize which signal occurs for these different circumstances so that he can take immediate response if an acute myocardial infarction is indicated but can take a non-emergency response if progression of the narrowing of a stenosis or some other less critical condition is indicated. It should be understood that other distinctly different audio alarm patterns could be used for different arrhythmias such as atrial fibrillation, atrial flutter, PVC's, PAC's, etc. A capability of the physician's programmer 68 of FIG. 1 would be to program different alarm signal patterns, enable or disable detection and/or generation of associated internal/external alarm signals in the cardiosaver for any one or more of these various cardiac events. Also, the intensity of the audio alarm, vibration or electrical tickle alarm could be adjusted to suit the needs of different patients. In order to familiarize the patient with the different alarm signals, the programmer 68 of the present invention would have the capability to turn each of the different alarm signals on and off.

FIG. 2 is a block diagram of the cardiosaver 5 with primary battery 22 and a secondary battery 24. The secondary battery 24 is typically a rechargeable battery of smaller capacity but higher current or voltage output than the primary battery 22 and is used for short term high output components of the cardiosaver 5 like the RF chipset in the telemetry sub-system 46 or the vibrator 25 attached to the alarm sub-system 48. An important feature of the present invention cardiosaver is the dual battery configuration where the primary battery 22 will charge the secondary battery 24 through the charging circuit 23. The primary battery 22 is typically a larger capacity battery than the secondary battery 24. The primary battery also typically has a lower self discharge rate as a percentage of its capacity than the secondary battery 24. It is also envisioned that the secondary battery could be charged from an external induction coil by the patient or by the doctor during a periodic check-up.

The electrodes 14 and 17 connect with wires 12 and 15 respectively to the amplifier 36 that is also connected to the case 11 acting as an indifferent electrode. As two or more electrodes 12 and 15 are shown here, the amplifier 36 would be a multi-channel or differential amplifier. The amplified electrogram signals 37 from the amplifier 36 are then converted to digital signals 38 by the analog-to-digital converter 41. The digital electrogram signals 38 are buffered in the First-In-First-Out (FIFO) memory 42. Processor means shown in FIG. 2 as the central processing unit (CPU) 44 coupled to memory means shown in FIG. 2 as the Random Access Memory (RAM) 47 can process the digital electrogram data 38 stored the FIFO 42 according to the programming instructions stored in the program memory 45. This programming (i.e. software) enables the cardiosaver 5 to detect the occurrence of a cardiac event such as an acute myocardial infarction.

A clock/timing sub-system 49 provides the means for timing specific activities of the cardiosaver 5 including the absolute or relative time stamping of detected cardiac events, calculation of heart-rate, and the provision of scheduled monitoring-operations. The clock/timing sub-system 49 can also facilitate power savings by causing components of the cardiosaver 5 to go into a low power standby mode in between times for electrogram signal collection and processing. Such cycled power savings techniques are often used in implantable pacemakers and defibrillators. In an alternate embodiment, the clock/timing sub-system can be provided by a program subroutine run by the central processing unit 44.

In an advanced embodiment of the present invention, the clock/timing circuitry 49 would count for a first period (e.g. 20 seconds) then it would enable the analog-to-digital converter 41 and FIFO 42 to begin storing data, after a second period (e.g. 10 seconds) the timing circuitry 49 would wake up the CPU 44 from its low power standby mode. The CPU 44 would then process the 10 seconds of data in a very short time (typically less than a second) and go back to low power mode. This would allow an ‘on’/‘off’ duty cycle of the CPU 44, which often draws the most power, of less than 2 seconds per minute while actually collecting electrogram data for 20 seconds per minute.

In a preferred embodiment of the present invention the RAM 47 includes specific memory locations for 4 sets of electrogram segment storage. These are the recent electrogram storage 472 that would store the last 2 to 10 minutes of recently recorded electrogram segments so that the electrogram data occurring just before the onset of a cardiac event can be reviewed at a later time by the patient's physician using the physician's programmer 68 of FIG. 1. For example, the recent electrogram storage 472 might contain eight 10-second long electrogram segments that were captured every 30 seconds over the last 4 minutes.

The baseline electrogram memory 474 would provide storage for baseline electrogram segments collected at preset times over one or more days. For example, the baseline electrogram memory 474 might contain 24 baseline electrogram segments of 10 seconds duration, one from each hour for the last day, and information abstracted from these baselines.

A long term electrogram memory 477 would provide storage for electrograms collected over a relatively long period of time. In the preferred embodiment, every ninth electrogram segment that is acquired is stored in a circular buffer, so that the oldest electrogram segments are overwritten by the newest one.

The event memory 476 occupies the largest part of the RAM 47. The event memory 476 is not overwritten on a regular schedule as are the recent electrogram memory 472 and baseline electrogram memory 474 but is typically maintained until read out by the patient's physician with the programmer 68 of FIG. 1. When a cardiac event is detected by the CPU 44, all (or part) of the entire contents of the baseline and recent electrogram memories 472 and 474, or statistical summaries of these data, would typically be copied into the event memory 476 so as to save the pre-event data for later physician review.

In the absence of the occurrence of cardiac events, the event memory 476 could be used temporarily to extend the recent electrogram memory 472 so that more data (e.g. every 10 minutes for the last 12 hours) could be held by the cardiosaver 5 of FIG. 1 to be examined by a medical practitioner at the time a patient visits. This would typically be overwritten with pre- and post-event electrogram segments following a detected event.

An example of use of the event memory 476 is a SEE DOCTOR alert which causes the saving of the last data segment that triggered the alarm and the baseline data used by the detection algorithm in detecting the abnormality. An EMERGENCY ALARM would save the sequential data segments that triggered the alarm, a selection of other pre-event electrogram segments, or a selection of the 24 baseline electrogram segments and post-event electrogram segments. For example, the pre-event memory would have baselines from −24, −18, −12, −6, −5, −4, −3, −2 and −1 hours, recent electrogram segments (other than the triggering segments) from −5, −10, −20, −35, and −50 minutes, and post-event electrogram segments for every 5 minutes, for the 2 hours following the event, and for every 15 minutes thereafter. These settings could be pre-set or programmable. When more than 1 electrode is available, the post-event data which is subsequently stored could be limited to the electrode at which the event was most strongly detected in order to provide efficient storage and enable a longer recording than would occur using multiple channels. Alternatively, post-event data could be expanded from 1 electrode to a set of 2 or more electrodes in order to provide a more thorough record of post-event cardiac condition.

The RAM 47 also contains memory sections for programmable parameters 471 and calculated baseline data 475. The programmable parameters 471 include the upper and lower limits for the normal and elevated heart rate ranges, and physician programmed parameters related to the cardiac event detection processes stored in the program memory 45. The calculated baseline data 475 contain values of characteristics of the data that are defined by the detection parameters extracted from the baseline electrogram segments stored in the baseline electrogram memory 474. Calculated baseline data 475 and programmable parameters 471 would typically be saved to the event memory 476 following the detection of a cardiac event. The RAM 47 also includes patient data 473 that may include the patient's name, address, telephone number, medical history, insurance information, doctor's name, and specific prescriptions for different medications to be administered by medical practitioners in the event of different cardiac events.

It is envisioned that the cardiosaver 5 could also contain pacemaker circuitry 170 and/or defibrillator circuitry 180 similar to the cardiosaver systems described by Fischell in U.S. Pat. No. 6,240,049.

The alarm sub-system 48 contains the circuitry and transducers to produce the internal alarm signals for the cardiosaver 5. The internal alarm signal can be a mechanical vibration, a sound or a subcutaneous electrical tickle or shock.

The telemetry sub-system 46 with antenna 35 provides the cardiosaver 5 the means for two-way wireless communication to and from the external equipment 7 of FIG. 1. Existing radiofrequency transceiver chip sets such as the Ash transceiver hybrids produced by RF Microdevices, Inc. can readily provide such two-way wireless communication over a range of up to 10 meters from the patient. It is also envisioned that short range telemetry such as that typically used in pacemakers and defibrillators could also be applied to the cardiosaver 5. It is also envisioned that standard wireless protocols such as Bluetooth and 802.11a or 802.11b might be used to allow communication with a wider group of peripheral devices.

A magnet sensor 190 may be incorporated into the cardiosaver 5. An important use of the magnet sensor 190 is to turn on the cardiosaver 5 on just before programming and implantation. This would reduce wasted battery life in the period between the times that the cardiosaver 5 is packaged at the factory until the day it is implanted.

The cardiosaver 5 might also include an accelerometer 175. The accelerometer 174 together with the processor 44 is designed to monitor the level of patient activity and identify when the patient is active. The activity measurements are sent to the processor 44. In this embodiment the processor 44 can compare the data from the accelerometer 175 to a preset threshold to discriminate between elevated heart rate resulting from patient activity as compared to other causes.

Additional details regarding a possible implementation of the cardiosaver 5 may be found in Ser. No. 11/594,806, filed November 2006, entitled “System for the Detection of Different Types of Cardiac Events.”

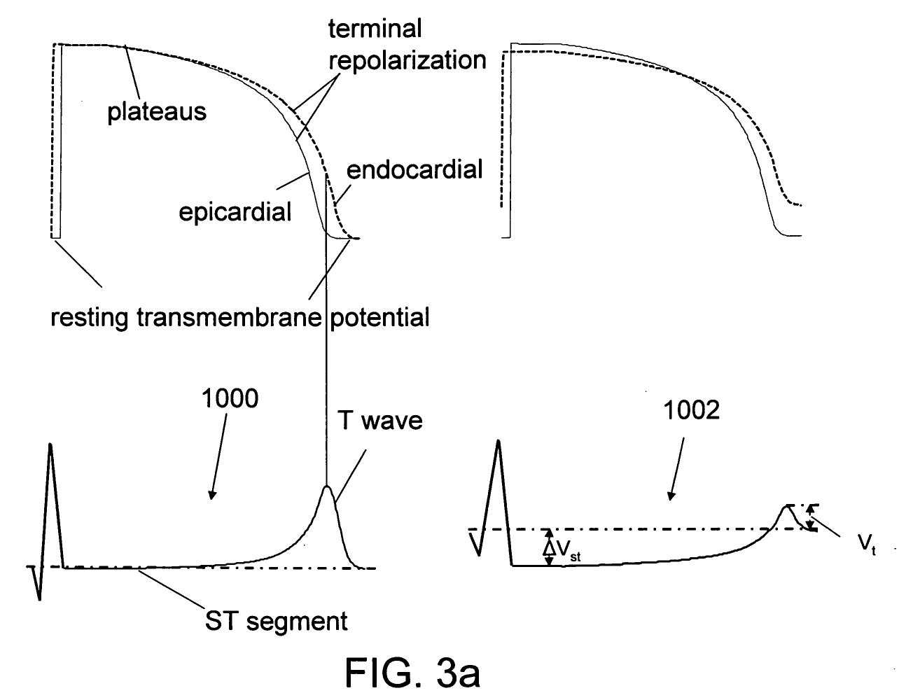

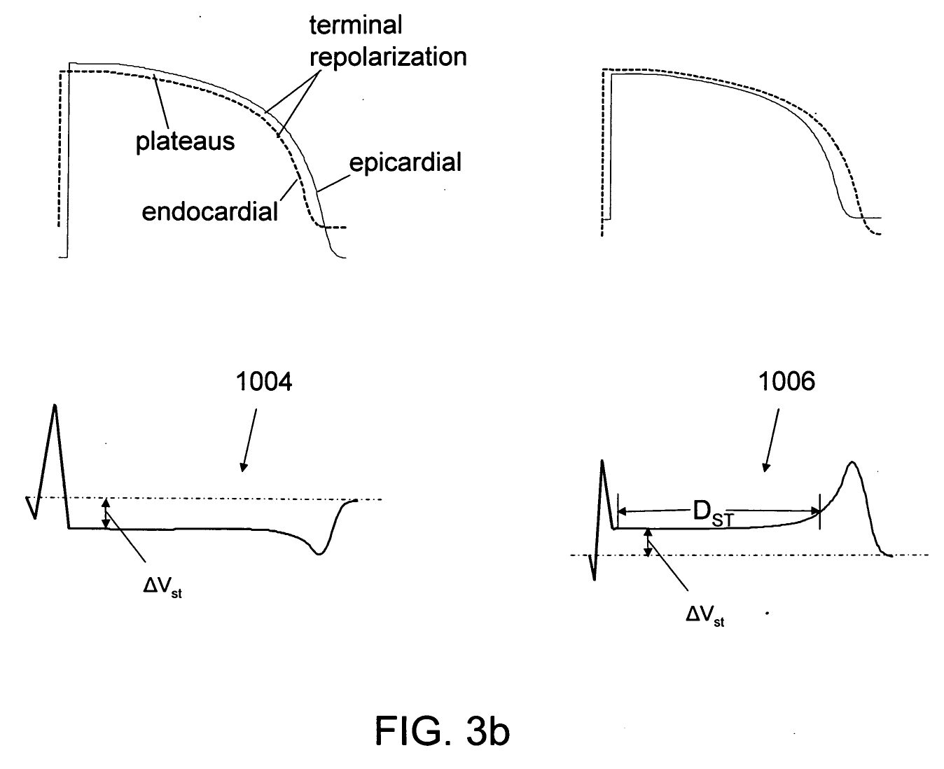

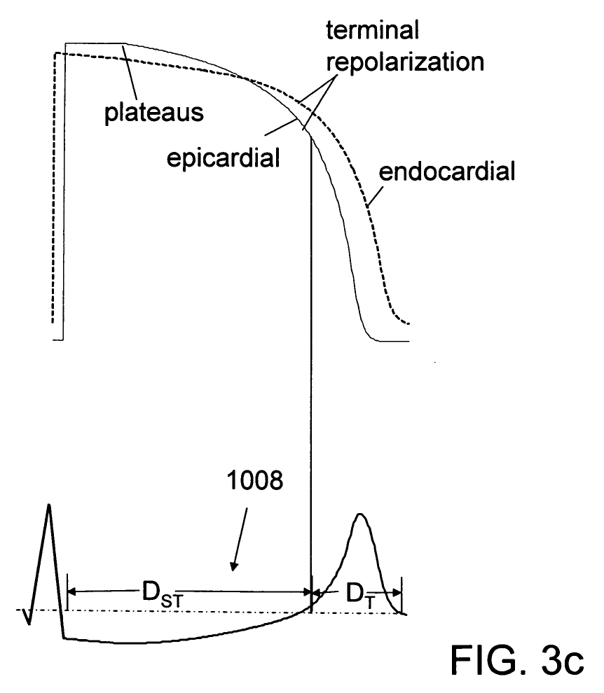

According to one embodiment of the present invention, a program residing in program memory 45 (FIG. 2) applies different tests for ischemia depending on the categorization of an electrogram. Example waveforms from some of the different electrogram categories are shown in FIGS. 3a-3c. In FIGS. 3a-3c, except as otherwise specified, the hypothetical epicardial (line) and endocardial (dashed line) action potentials which may underlie electrogram shapes are shown at the top of the figures and corresponding electrograms are shown at the bottom of the figures. In the electrograms of FIGS. 3a-3c, the ST and T wave portions were obtained by subtracting the simulated endocardial potential from the simulated epicardial potential. The modeled electrograms which result from this subtraction are similar in shape to those that may be expected from a real lead configuration in which the electrode 14 of lead 12 (FIG. 1) is placed within the heart and the electrode 13 is outside the heart, and the lead voltage is defined as the voltage at electrode 13 (i.e. outside the heart) minus the voltage at electrode 14 (i.e. inside the heart). It will be understood that all references to polarity (i.e. positive or negative voltages) in the discussion below are based on this choice.

Electrograms are determined by a complex distribution of transmembrane cardiac potentials. The inventors believe that many important features of electrograms which are associated with ischemia may be analyzed by comparing two types of gradients: transmural (e.g. endocardial to epicardial) and intra-layer (e.g. the gradient across the endocardium or the gradient across the epicardium.) Although both types of gradients may be important for generating an electrogram, a comparison of the transmural gradients is convenient. Thus, as mentioned, FIGS. 3a-3c show simulated transmural potential differences that would result in the electrogram shapes that may be recorded in an actual patient.

In the electrograms shown in FIGS. 3a-3c, the reference voltage (horizontal dash-dot line) is calculated as the average voltage across the PQ segment, which in turn is hypothesized to result from the difference in resting transmembrane potentials between cells. In a healthy person, there is generally no difference in resting transmembrane potentials. However, ischemic cells have different resting transmembrane potentials than healthy cells, which drive current flow and voltage drops during the PQ segment. As is known in the art, current flow patterns during the PQ or TQ segment provide a direct picture of the distribution of ischemic and healthy cells, uncomplicated by activation and repolarization sequences.

Turning to FIG. 3a, electrogram 1000 is what may be expected in a healthy patient. In this simulation data, at the top left of the figure, there is no resting transmembrane potential difference between the cells. The ST segment is basically isoelectric because the corresponding endocardial and epicardial action potential plateau voltages are equal. The epicardium terminally repolarizes before the endocardium, resulting in a positive T wave.

On the right side of FIG. 3a an electrogram 1002 is shown that may occur in the context of subendocardial ischemia. In this case, the endocardial action potential, which is either ischemic or strongly electrically coupled to ischemic cells, has a greater resting transmembrane potential but lower peak (or average) amplitude of its plateau region than the (relatively) non-ischemic epicardial cell. This drives current flow during the ST segment that is opposite to the current flow that occurs during the (reference) PQ segment and results in a negative ST deviation ΔV_{St}. Furthermore, the endocardial cell repolarizes earlier compared to the healthy case, which reduces the amplitude of the T wave. In cases where the endocardial plateau has a relatively steeper slope than the healthy endocardial plateau, ST depression may be downsloping, which is sign of more severe ischemia.

The ST segment depression shown in electrogram 1002 may also be recorded from a subendocardial electrode outside of an ischemic region at relatively higher heart rates (e.g., greater than 120 beats per minute). In this case, various activation/repolarization sequence effects can cause most or all of the endocardium, including the non-ischemic subendocardium, to be relatively more repolarized during the early portions of the ST segment. As the ST segment progresses, a waveform from an ischemic subendocardial region would be expected to become relatively more positive than a waveform from a non-ischemic subendocardial region. The epicardium will tend to “catch up” to the non-ischemic subendocardium, reducing or eliminating the transmural gradient that tends to cause early ST segment positive potentials in the ischemic region. This would be counteracted by the tendency of the non-ischemic area to have a negative potential compared to the ischemic subendocardial region. In an embodiment in which two subendocardial electrodes are available, a waveform derived from a lead defined by the two electrodes could provide additional information regarding the positioning of the electrodes with respect to the ischemic region(s).

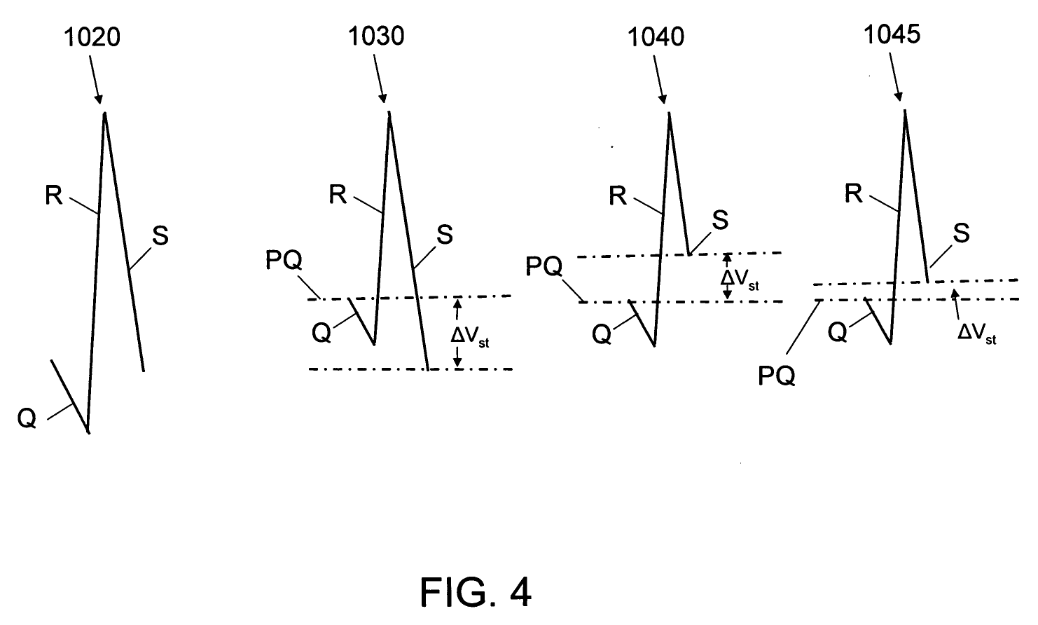

If ST depression is due to heart rate effects alone, and is not the result of any pathological condition, then the ST segment should be upsloping, and the Q wave amplitude should not decrease, as it does in the case of ischemia due to differences in resting transmembrane potential (“diastolic injury current”, see FIG. 4 and associated description). For torso surface electrocardiograms, clinicians have long examined the slope of the ST segment to help distinguish between normal and pathological causes of ST segment depression at higher heart rates.

A more severe example of subendocardial ischemia is indicated by electrogram 1004 in FIG. 3b (label top of FIGS. 3b and 3c as per 3A). In this case, the simulated endocardial cell repolarizes before the epicardial cell, which results in an inverted T wave in the corresponding electrogram 1004. Again, there is a negative ST deviation ΔVₛₜ. The endocardial electrode 14 may be either inside or outside of the ischemic region and the ischemic changes will still be detected because of the (believed) global nature of subendocardial ischemia. If the epicardial action potential curve is shifted a little to the left, a biphasic T wave (initially negative then positive) can occur.

Electrogram 1004 also exemplifies a waveform shape that may occur when the ischemia is transmural, the inner heart electrode 14 is within the ischemic region, and the indifferent electrode 13 represents a reasonably good ground during repolarization (e.g. in the upper left torso). In this case, the ST and T wave shifts do not result primarily (if at all) from transmural transmembrane voltage gradients but instead occur mostly (if not wholly) as a result of transmembrane voltage gradients between the transmural ischemic region and the non-ischemic regions.

Electrogram 1006 shows what may be expected in the case of transmural ischemia when the inner heart electrode 14 is outside of the ischemic region. In this case, the entire epicardium repolarizes earlier and has a smaller plateau than the non-ischemic portions of the inner heart. Thus, the T wave is positive (as in the normal case) but there is a positive ST deviation ΔVₛₜ. Furthermore, the duration of the ST segment (D_{ST}) is abnormally short because the epicardium is repolarizing abnormally early (for the given heart rate).

Electrogram 1008 in FIG. 3c shows a pattern that may occur in the context of transmural ischemia when the inner electrode 14 is within a (proximal) ischemic region, the indifferent electrode 13 represents a reasonably good ground during repolarization, and transmural ischemia may be occurring in another (distal from electrode) ischemic region. In this case, as before, the dashed action potential represents the activity of the ischemic area which surrounds the electrode 14. The other action potential (filled line) represents a composite; the plateau is from a non-ischemic subendocardial region, and the terminal repolarization segment is from the epicardium. During the ST segment, the electrode 14 is in an ischemic subendocardial region. A negative ST deviation ΔVₛₜ is due to gradients between the proximal ischemic region and non-ischemic subendocardium. During the T wave, since the transmural ischemia tends to cause the entire epicardium to repolarize earlier than normal, the T wave is large (positive).

The electrogram 1008 may occur in cases where the inner electrode 14 is within (or near) a chronically ischemic region that generally corresponds to electrogram 1002 (FIG. 3a), and a different region becomes transmurally ischemic. When the ischemia in the different region becomes transmural, the magnitude of ΔVₛₜ decreases (e.g. ΔVₛₜ is larger for electrogram 1008 compared to electrogram 1002). This occurs because the epicardium (due to the transmural ischemia) is now “pulling” the inner heart's potential (including electrogram 1004) toward ST elevation. This shift begins to cancel the ST depression resulting from the gradient between the ischemic inner heart region and non-ischemic inner heart region. Stated another way, the electrogram 1008 may be thought of as a composite of waveforms 1002 and 1006 (transmural ischemia). Considering waveform 1002 as a baseline and subtracting it from waveform 1008 tends to yield a waveform more akin to 1006.

Since different cardiac event signatures putatively have differing underlying causes, the classification of electrograms, as a function of their underlying physiological processes, allows more accurate evaluation of their medical severity and relevance. By applying tests to the electrograms which are selected based upon the probable causes of different features, the features can be assessed in an improved manner. This strategy improves diagnostic validity of the detected events, since inappropriate tests, or thresholds used by these tests, are not applied to features of the electrogram.

Ischemia is also known to change the QRS complex. The manner in which QRS changes are incorporated into the inventive ischemia detection scheme will be described with reference to QRS complexes shown in FIG. 4, which are the type of complexes that may be especially expected when the inner heart electrode 14 is within the ischemic region, and the indifferent electrode 13 is within the torso. Although the QRS complexes are described with reference to this orientation, the principles outlined below are applicable to a wide variety of electrode configurations.

The QRS 1020 represents a normal QRS complex. The Q wave downstroke occurs as an activation wavefront approaches the electrode 14. The R wave upstroke occurs as the region surrounding the electrode 14 depolarizes. The S wave occurs as the wavefront moves away from the region surrounding the electrode 14. The end of the S wave represents the point in time when all cells within the heart have been reached by the activation wave. If the heart is isoelectric during the ST segment and all cells have the same resting potential, then the voltage at the end of the S wave is equal to the baseline voltage before the start of the Q wave. Thus, Q+R+S should approximate a value of zero when the heart is functioning normally, and should deviate away from zero in differential manners as a function of different types of disorders.

Waveform 1030 is QRS complex that corresponds to a case of ST segment depression. In this case, Q+R+S<0. Waveform 1040 is QRS complex that corresponds to a case of ST segment elevation. In this case, Q+R+S>0. The sum of the Q, R and S waves can serve as a proxy that indicates ST segment elevation or depression.

Furthermore, a reduced Q wave amplitude/slope suggests ischemia in the region that surrounds the electrode 14 and/or ischemia in the upstream region (from which the activation wave propagates to the electrode 14 region). Reduced R wave amplitude and/or slope suggests ischemia in the region that surrounds the electrode 14. Finally, reduced S wave amplitude and/or slope suggests ischemia in the downstream region (to which the wavefront propagates from the electrode 14 region). Prolongation of any of the Q, R and S wave durations may also indicate ischemia. Notching or slurring of QRS portions are also known to indicate the presence of ischemia.

For an electrode outside of an ischemic region, at high heart rates, heart rate effects above with regard to electrogram 1002 (FIG. 3a) could tend to cancel diastolic injury current effects, so that ST deviations are small even though ischemia is present. More particularly, the ST segment may tend to be low (due to heart rate effects) while the PQ segment may also tend to be low due to difference in resting transmembrane potentials between normal and ischemic cells. If the ST segment deviation is defined using the PQ segment as a baseline, this deviation may be small, as indicated by waveform 1045 in FIG. 4. Thus, to detect ischemia, it may be desirable to check Q wave amplitude alone as an additional test, and also heart rate dependent R wave upstroke (peak R−bottom Q) and S wave downstroke (peak R−bottom S).

Relatedly, prolongation of QRS duration with heart rate, and/or an increase in QRS duration in cases where there is a decrease in the QT interval, is a possible indicator of ischemia.

The reviewed electrogram features may all be used to classify the electrogram data as belonging to different categories or classes, and to constrain the analysis and evaluation of the electrogram based upon this classification. This method can offer a number of advantages, such as increasing the sensitivity and specificity of detecting cardiac events, decreasing the complexity of the algorithms which are used, and decreasing the number of statistical comparisons which are made for a particular electrogram segment. Rather than performing a test upon possible feature of the electrogram (e.g., testing the QRS duration, testing the R-wave amplitude, testing the sum of the QRS components, and testing the ST-deviation, etc.) the features which are examined can be made contingent upon classification tests. In one example, the QRS duration is not tested unless the test for the QT interval indicates a decrease in this measure which is in a specified range so as to classify the electrogram as belonging to a “short QT-interval” class. By only submitting electrograms of particular classes to a constrained number of tests, the advantages just described can be realized. Further, since the only tests which are performed are done so because other tests have already been met, spurious analysis of the data does not occur and will also serve to use less power from the implanted power source since these tests require processing from the system's CPU.

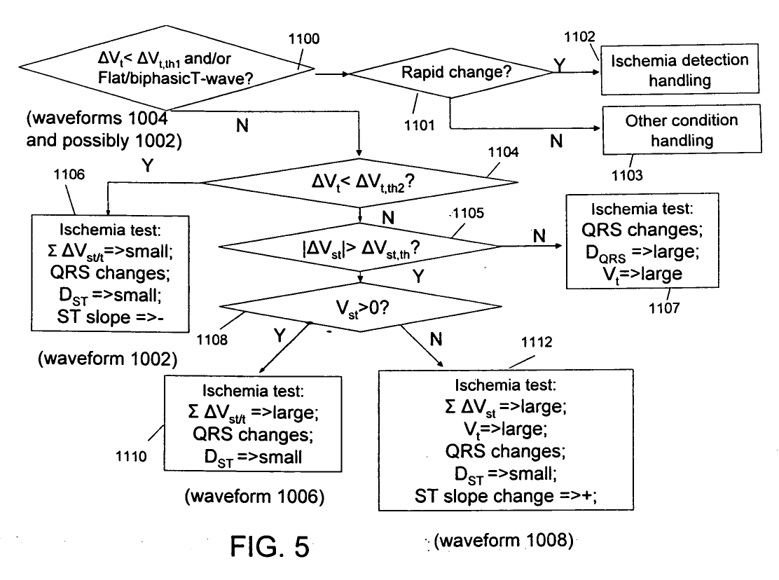

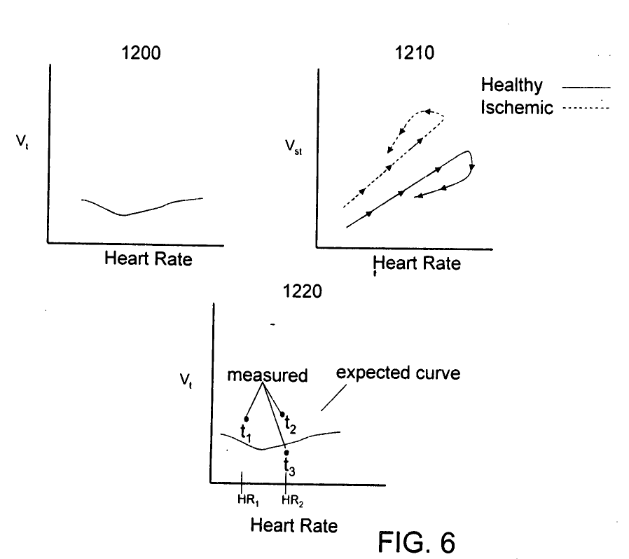

FIG. 5 is a flow chart of an ischemia detection routine according to the present invention. As will be mentioned, many ischemia test factors are heart rate dependent. Determination of heart rate dependent test thresholds will be described with reference to FIG. 6.

The flow chart shown in FIG. 5 represents a hierarchical diagnostic model that serves to constrain various criteria to specific situations or classes of disorder. The earlier stages in the method are used to broadly detect pathology, using a gross diagnostic criteria. The later stages divide the data into two or more distinct classes, each of which is analyzed in a unique manner according to one or more criteria (termed class diagnostic criteria).

Turning to FIG. 5, in step 1100, three tests are initially applied to the T-wave. Firstly, the T wave amplitude (ΔVₜ) is compared to a threshold (ΔV_{t,th1}). The threshold ΔV_{t,th1} is preferably set to a low value (i.e. small positive value or negative value) to capture cases of severe ischemia. Secondly, a flat or inverted T wave suggests the possibility of severe ischemia (e.g. waveform 1004 and perhaps 1002). Thirdly, a biphasic T-wave may indicate ischemia as described above and will be detected in step 1100. If ΔVₜ<ΔV_{t,th1}, or if the T-wave is too flat, or if the T-wave is bi-phasic, then an adverse cardiac condition is detected and the routine moves to block 1101, which checks the rate of change in the T wave amplitude. A flat T-wave is not necessarily specific for ischemia whereas if this occurs in addition to rapid changes in T wave amplitude with respect to time (at a fixed heart rate) then ischemia is more likely. If the change has been rapid, then control passes to block 1102, where ischemia detection is handled (e.g. patient alerting etc.). Otherwise, control passes to block 1103, which handles other types of less immediately serious conditions (e.g. a milder form of patient alerting may make sense.) Block 1103 may also implement additional types of condition detecting. For example, it may check for a very large amplitude negative T wave, which is suggestive of hyperkalemia.

It will often be desirable to detect ischemia only if many electrogram segments, heart beats, averaged beats, or other measurement of cardiac activity, indicate an ischemic condition. In this case, ischemia is not detected directly in block 1102. Rather, a counter may be incremented and ischemia may be detected only when the counter reaches a threshold value within a specified duration. The counter can be zeroed periodically so that only recent events are included in the count. This threshold value may be static or a function of the outcome of certain operations (e.g. self-norm) or of ischemia tests. If ΔVₜ>=ΔV_{t,th1}, then the routine moves to block 1104.

In step 1104, T wave amplitude (ΔVₜ) is compared to a threshold (ΔV_{t,th2}). This step is designed to separate cases of late or chronic subendocardial ischemia (waveform 1002) from transmural ischemia (1006, 1008). This step therefore acts to classify the electrogram into one of 2 categories (chronic subendocardial ischemia and transmural ischemia) and to perform unique tests according to this classification in order to detect cardiac events. The threshold ΔV_{t,th2} is preferably set to approximately the lower bound of the expected normal T wave amplitude. The threshold ΔV_{t,th2} can be adjusted by the algorithm according to the patient's heart rate.

If ΔVₜ<ΔV_{t,th2} then the routine moves to block 1106, where it applies an ischemia test appropriate for waveforms of the type 1002. This ischemia test is a function of four factors: (i) the sum (or integral) of waveform voltage over the entire ST and T segments (with negative voltages counting against positive voltages), with a smaller sum indicating an increased likelihood of ischemia; (ii) reduction in QRS amplitudes/slopes as described with reference to FIG. 4; (iii) small D_{ST}; and (iv) an analysis of the slope of the ST segment, with any negative slopes indicating a greater likelihood of ischemia. To some extent, the ST/T sum test includes information regarding the ST slope test (iv). Relatedly, although the ST/T sum test includes information regarding the duration of the T wave, D_{T}, a separate D_{T} test could also be included, with the smaller D_{T} indicating a greater likelihood of ischemia. In the figure, the values on the right side of the “=” symbol, in other words, “small”, “large”, “−”, “+”, all refer to threshold values which can be selected for the patient by a physician, which can be based upon self or population normative data, can be heart rate dependent, or can be otherwise selected in order to provide improved detection of cardiac events. However, in all of these cases the threshold is also dependent upon the category of the test. In other words, “small” in step 1106 can be selected to be a different value than “small” in step 1110, since these two steps are evaluations of different categories of electrogram. Similarly, “QRS changes” are changes whose magnitudes can be programmably selected according to the patient's condition, but can also be adjusted depending upon the electrogram category.

D_{ST} may be defined in different ways. D_{ST} may be defined as the point of maximum curvature which occurs after the onset of the ST segment and before the peak of the T-wave. The value of this maximum curvature provides a measure of the relative repolarization times of epicardial and endocardial cells, with greater curvature (and less symmetric T waves and longer D_{T}) indicating relatively earlier repolarization of epicardial cells.

The above ischemia test may be written as a function of the above waveform characteristics: f(c₁, c₂, c₃ . . . cᵢ), where the cᵢ are the waveform characteristics. The output of this function may be compared with a threshold to estimate whether ischemia is present. IMultivariate equations (and their coefficients) which are used to detect cardiac events such as ischemia can be selected and implemented based upon categorization of the electrogram data. Additionally, the thresholds can be adjusted based upon this categorization. Alternatively, each waveform characteristic cᵢ may have its own threshold tᵢ that is incorporated into the test function: f(c₁−t₁, c₂−t₂, c₃−t3, c₄−t₄ . . . ), the output of which may then be compared to another threshold. Further, the thresholds for various characteristics are preferably heart rate dependent and may be determined by a patient stress test, as described with reference to FIG. 6 for the case of ST shifts. All of the tests described below with reference to FIG. 5 may be formulated in this manner (i.e. f(c₁−t₁, c₂−t₂, c₃−t₃, c₄−t₄ . . . ).

Other sensed data (including data from non-electrical sensors) may be used both to help classify a particular electrogram, and as part of the data analyzed during a test designed to detect a cardiac event such as an ischemia test.

Returning to block 1104, if ΔVₜ>=ΔV_{t,th2}, then the routine moves to block 1105, where it checks ST segment amplitude. Preferably, this test also weights the rapidity of any ST segment changes, with more rapid changes indicative of ischemia and therefore increasing the likelihood of the step passing control to block 1107. For a beat that does exhibit ST changes according the chosen criteria, control passes to block 1107, which examines the beat for QRS changes. QRS duration (D_{QRS}) is preferably examined. Because there have not been any significant ST changes (as determined in block 1105), the QRS test implemented in block 1107 may impose relatively strict criteria to trigger detection of an ischemic event. A large T wave and/or rapid changes in T wave amplitude may also be examined.

Returning to block 1105, if an ST change has been detected, block 1105 passes control to block 1108, which checks if the waveform exhibits ST elevation by direct examination of the ST segment or by examination of an indirect proxy for ST elevation, such as the QRS test described with reference to FIG. 4.

If ST elevation is detected in step 1108, then the electrogram data is classified in the ‘waveform 1006’ category and the routine moves to block 1110, where it applies an ischemia test appropriate for waveforms like waveform 1006. The ischemia test is a weighted function of three factors: (i) the sum (or integral) of waveform voltage over the entire ST and T segments, with a larger sum indicating an increased likelihood of ischemia; (ii) reduction in QRS amplitudes/slopes as described with reference to FIG. 4, especially reductions in S wave slope; and (iii) small D_{ST}.

If ST elevation is not detected in step 1108, the routine moves to block 1112, where it applies an ischemia test appropriate for cases of chronic subendocardial ischemia. The ischemia test is a weighted function of six factors: (i) the sum (or integral) of waveform voltage over the ST segment (with negative voltages counting against positive voltages), with a positive change indicating an increased likelihood of ischemia; (ii) T-wave amplitude Vₜ, with larger values indicating a greater likelihood of ischemia; (iii) reduction in QRS amplitudes/slopes as described with reference to FIG. 4, especially S wave slope; (iv) small D_{ST}; (v) an analysis of the slope of the ST segment, with a change toward positive shapes indicating a greater likelihood of ischemia.

As mentioned above, changes in T wave amplitude over short time periods may be indicative of ischemia. More generally, an analysis of the change of various electrogram characteristics (e.g. T wave amplitude) over time conveys additional information regarding the state of the patient. Thus, for every test factor described with reference to FIG. 5, it may be desirable to check not only absolute values of electrogram characteristics, but the rate of change in those characteristics over time, where the threshold for rate of change can be differently set for different classification categories.

One possible difficulty with detecting such changes is that various electrogram characteristics change as a function of heart rate. For example, in a normal, young person, T wave amplitude (as measured from certain surface leads) generally decreases with moderate exercise and then increases at maximal exercise, as shown in plot 1200 in FIG. 6. The plot 1210 of FIG. 6 is adapted from Noninvasive Electrocardiology in Clinical Practice, Zareba, Maison-Blanche and Locati eds (Futura, 2001), and shows ST segment deviation (as measured from surface leads) as a function of heart rate for normal (solid line) and ischemic (dashed line) subjects. The slope corresponding to the ischemic subject is greater than the slope for the healthy subject. Both curves exhibit hysteresis (the arrows indicate the direction of heart rate changes), which is counterclockwise in the case of the ischemic subject but clockwise in the case of the healthy subject. There is also a hysteresis for T wave amplitude that likely differs between healthy and ischemic subject.

In any event, if electrogram characteristic/heart rate curves can be constructed for a subject by tracking these characteristics over time and compared with a baseline or “healthy” curve for that subject, and additional ischemia test could involve comparison of the evolving curve with the baseline curve.

The U wave is another heart rate dependent feature. U wave magnitude is inversely correlated with heart rate. Thus, if the heart rate is low, then an examination of U wave amplitude may yield information regarding the presence of ischemia. One experiment involving intracoronary electrograms (Use of intracoronary electrocardiography for detecting ST-T, QTc, and U wave changes during coronary balloon angioplasty, Safi et al., Heart Dis, 2001; 3(2):73-6.), suggests that U wave amplitude, as measured by an intracoronary electrode in the area of the ischemic region, increases with greater ischemia. However, it is also possible that in certain circumstances, U wave amplitude may decrease with increasing severity of ischemia. Thus, it may be desirable to check for changes in U wave amplitude from a baseline value at low heart rates.

A different test, as mentioned above, is to detect the rate of change of a characteristic (e.g. T wave amplitude) over time. FIG. 1220 shows a normal or expected T wave amplitude curve (solid line) for a subject, which may be patient specific. The filled dots represent measurements made at times t₁, t₂ and t₃ respectively. The later times, t₂ and t₃, are assumed to occur during an ischemic event. If the system is applying a rate of change in Vₜ test just after t₂, a direct calculation of (Vₜ(t₂)−Vₜ(t₁))/(t₂−t₁) would tend to understate the rise in T wave amplitude because there is an expected rise due simply to the different heart rates at t₁, and t₂, respectively. Instead, the actual rate of change should be compared to the expected rate of change, (Vₜ(HR₂)−Vₜ(HR₁))/(t₂−t₁). For example, the expected rate of change may be subtracted from the actual rate of change to arrive at an adjusted rate of change characteristic.

If early subendocardial ischemia persists and the heart rate at times t₂ and t₃ is the same, then (Vₜ(t₃)−Vₜ(t₂))/(t₃−t₂) will be the adjusted rate of change characteristic since (Vₜ(HR₂)−Vₜ(HR₂)=0).

There are different alternatives for handling the possibility of hysteresis in the parameter/heart rate curves. If measurements are being taken over a sufficiently small time scale, then the hysteresis can be directly tracked and compensated for.

More general tests that analyze an entire time ordered trajectory of V, measurements can be constructed.

Making the heart rate curves patient and circumstance specific can improve ischemia test sensitivity/specificity. For example, if a patient has just undergone a stent implantation, his/her ST segment deviations would be expected to resolve (move toward an isoelectric ST segment) over time. This progression corresponds to a family of ST/heart rate curves. The exact member of this family to select as the “normal” curve at a particular time could be programmed as a function of time from the stenting procedure, or can be selected based on the (slowly evolving) baseline ST deviation. Furthermore, since positive shifts in ST deviation are expected, the ischemia threshold for ST shifts could be set to a greater value for positive shifts than negative shifts.

All static thresholds (e.g. D_{ST}) mentioned with respect to the ischemia detection routine described with respect to FIG. 5 are preferably determined according to an expected heart rate curve.

FIG. 5 illustrated a routine for detecting ischemia by sequentially analyzing various electrogram characteristics as a means of categorizing electrograms. An alternate embodiment of the present invention, which does not rely on sequential processing to categorize waveforms, involves construction of a single (non-linear, discontinuous, multivariable) function/mapping that effectively implements the sequential logic shown in FIG. 5. For example, at least one lookup table may be used wherein the rows are parameters and the columns are ranges of values. According to one embodiment, unless the parameter for the first row of the lookup table is within the ranges defined in a particular column, additional rows of the column are not evaluated. Alternatively, multiple columns could be checked simultaneously.

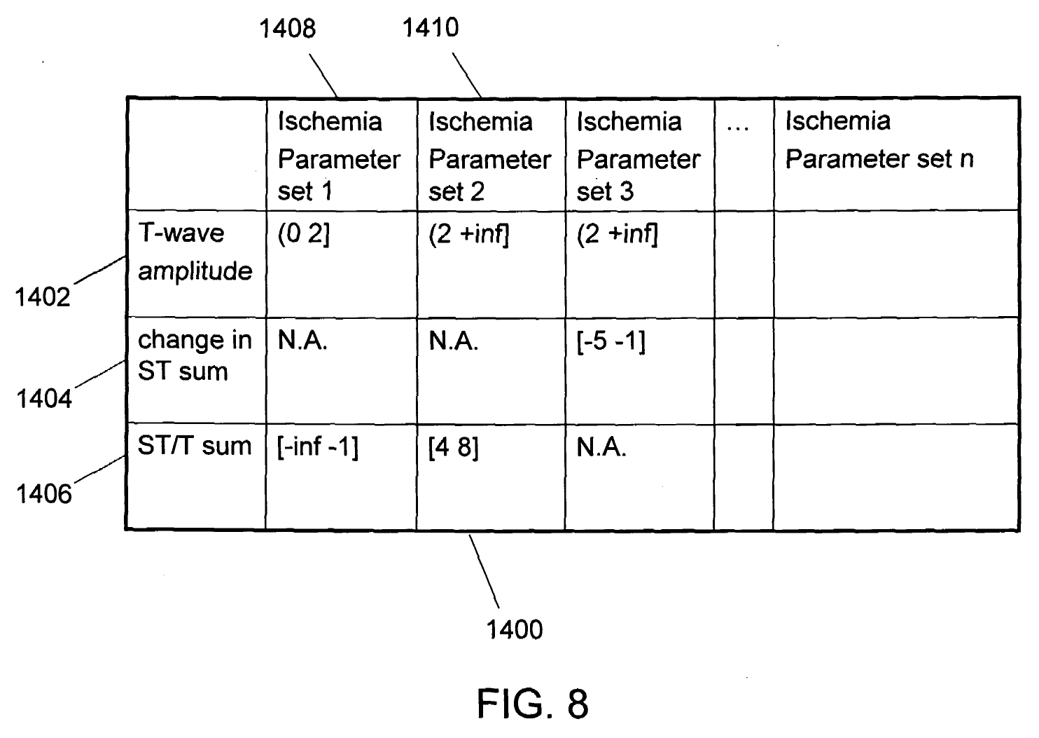

FIG. 8 is an example of such a table lookup scheme. A table 1400 has three rows, 1402, 1404 and 1406, that contain entries for T wave amplitude, ST sum, and ST/T sum, respectively. Each column in the table corresponds to a set of parameter value ranges that is associated with a type of electrogram category that will differentially be evaluated and will trigger detection of ischemia when satisfied, i.e., the criteria in one column of the table are compared with test data and the results are combined with the logical AND operator. For example, assuming that V_{t,th1}=0 and V_{t,th2}=2 (see blocks 1100 and 1104 in FIG. 5), column 1408 corresponds to the case of a T wave amplitude that is less than V_{t,th2}, but greater than V_{t,th1}, so that the ischemia test in block 1106 is implemented. A bracket indicates inclusion of the range end point whereas a parenthesis indicates exclusion of the end point. In practice, −inf (−infinity) can be bounded at some very large magnitude negative number. For ease of illustration, only the ST/T sum portion of the test is illustrated in the table. An ST/T sum of −1 or less will result in ischemia detection.

Column 1410 corresponds to block 1110 (FIG. 5), which corresponds to ST elevation, and column 1412 (this is not in FIG) corresponds to block 1112 (FIG. 5), ST depression with a relatively large T wave. Ischemia is detected if any of the columns (logical OR operation) are positive for ischemia. More than one column may be positive for ischemia (this is not true in FIG. 5 strategy where only one box is able to be true) because the ischemia tests (e.g. in blocks 1106, 1107, 1110 and 1112 in FIG. 5) are preferably implemented with OR logic, as previously described.

The structures shown in FIGS. 5 and 8 allows certain parameters (e.g. T wave amplitude) to be used very flexibly. Continuing with the example of T wave amplitude, not only can T wave amplitude be indicative of ischemia if it is either too high or too low, but the degree to which it is too high or too low can also be taken into account. For example, if there are no ST changes and block 1107 (FIG. 5) is applied, then the magnitude of T wave amplitude required to trigger ischemia may be greater than if ST changes are also observed, in which case block 1112 contains the appropriate ischemia test. This analysis assumes that the ischemia tests in blocks 1107 and 1112 can be positive based on T wave amplitude alone, i.e. T wave amplitude is tested against a threshold and the result is OR'd with whatever other subtests are performed, some of which may be contingently invoked based upon the characteristics (is this what you mean?) of T wave amplitude.

In addition, it also allows certain features to be included in an ischemia test or ignored, depending on the context. For example, the entire T wave is preferably examined in block 1110 (FIG. 5) whereas only the T wave amplitude is preferably examined in block 1112 (FIG. 5).

The ischemia detection schemes described with reference to FIGS. 5 and 8 may be viewed as functions (F(x)) that map heart signal feature values (vector x) to a cardiac state (e.g. F(x)=0 or 1, where 1 means an ischemic cardiac event is detected and 0 means it is not detected. If the hierarchical scheme shown in FIG. 5 is employed, only one function F(x) is computed for a given electrogram portion that is being tested for ischemia. For example, if the ischemia test in block 1112 (FIG. 5) is being applied,

$$F ( x ) = ( \overline{Δ {V}_{t} < Δ {V}_{t , th 1}} ) * ( \overline{Δ {V}_{t} < Δ {V}_{t , th 2}} ) * ( \vert Δ {V}_{st} \vert > Δ {V}_{st , th} ) * ( {V}_{st} > 0 ) * {f}_{112} ,$$

where f₁₁₂ is the (sub) function computed in block 1112 which has a binary output (1=ischemia is present), the relational operators <and > return binary values, and multiplication operator * corresponds to the logical AND operation. The particular function F(x) that actually is computed preferably depends on classification of the electrogram data, as in FIG. 5. In theory it would be possible to compute all possible functions F and detect ischemia if the value of any of them is 1, but this would not be the preferable embodiment since this is more computationally complex and obviates a number of the advantages of the described method.

Returning to the above example regarding T wave amplitude, which is compared to different thresholds depending on whether ST changes are present, a function F₁(x) corresponds to the path through the FIG. 5 hierarchy up to and including block 1112 while another function F₂(x) corresponds to the path through the FIG. 5 hierarchy up to and including block 1107. The functions F₁(x) and F₂(x), respectively, involve the application of different thresholds to T wave amplitude (through the subfunctions f₁₁₁₂ and f₁₁₀₇, respectively).

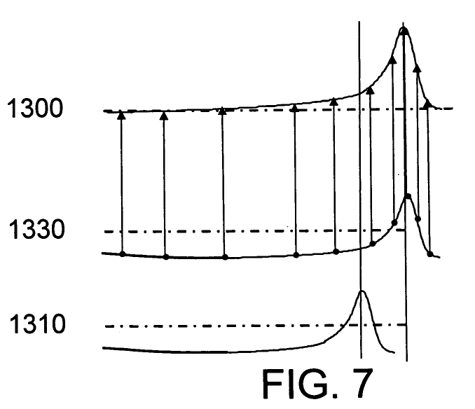

Yet another alternate embodiment will be described with reference to FIG. 7, which relies on analyzing derived measures of waveform characteristics, as opposed to the waveform characteristics themselves. FIG. 7 shows an expected (heart rate dependent) ST/T segment or ‘ST/T template’ 1300 and a measured electrogram 1310. To compare the measured electrogram 1310 with the template segment 1300, the measured electrogram 1310 is time-warped so that it matches to the expected ST/T segment. One manner of performing such warping is to first scale the time axis of the measured electrogram 1310 by a scaling factor (t_{sc}) so that the peak of its T wave coincides with the peak of the expected segment 1300 T wave, resulting in waveform 1330. Next, a number of splines defined by control points (filled circles in waveform 1330) may be fitted to the time scaled waveform 1330. The splines may then be transformed so that the scaled waveform 1330 best matches the waveform 1300 according to certain criteria (e.g. least squares error). These transformation parameters, along with the temporal transformation scaling parameter t_{sc}, enable a comparison of waveform 1310 with waveform 1300. A function/mapping of the transformation parameters may be constructed, thereby deriving an ischemia test that is based on an abstract characterization (i.e. the transformation parameters) of the waveform 1310.

According to yet another alternate embodiment, guard bands may be formed around a heart rate dependent template waveform. Waveforms that pass out of the guard bands may be classified as abnormal. Statistically-based guard-bands are preferable.

Although the above methods were described with reference to a lead comprising an electrode within the heart and outside the heart, the methods may be extended to the case of having all electrodes outside of the heart. Such electrodes may be epicardial, subcutaneous and/or on or near (but outside of) the body surface. In this case, an electrode pair that is oriented along the long axis of the heart can be treated in the same manner as the inner heart/outside inner heart electrode pair, since current flow along this axis reflects endocardial to epicardial current flow.

Many different types of electrode schemes may prove advantageous. For example, one scheme involves a first electrode inside the heart, a second electrode on or near the epicardium, and a third electrode in a remote location that acts as a ground. In these cases, the information from one lead may be used to help classify another lead, and/or the ischemia tests for all the leads may be combined in a single ischemia test, as is done for some existing multi-surface lead ischemia detection schemes.

The above methods described a particular example in which ischemic waveforms are distinguished from healthy waveforms. However, the classification approach described above may be used to distinguish ischemic changes from non-ischemic changes caused by some other pathology (e.g. hyperkalemia), or simply to classify (diagnose) other pathological changes associated with various types of cardiac abnormalities.

It may be desirable to implement computationally expensive procedures (e.g. Fast Fourier Transforms or ‘FFTs’) in various steps of FIG. 5. For example, it may be desirable to use an FFT to detect QRS spectral signatures, so that changes in spectral energy can be quantitatively assessed. In this case, an alternative to requiring the cardiosaver 5 to perform the detailed calculations would involve having the cardiosaver 5 first perform relatively simpler tests that classify waveforms as ischemic, non-ischemic or possibly ischemic. In the last case, the waveform in question may be sent to an external system with greater computational resources to perform additional tests that resolve the putative existence of a cardiac event. Further, the external system may have access to additional information, such as an external 12 lead electrocardiogram, that it can analyze in conjunction with the internal data.

The hierarchical ischemia detection scheme illustrated with reference to FIG. 5 may be implemented by considering data from sources (e.g. a sensor that detects left ventricular end diastolic pressure) in addition to an electrogram. Non-electrical sensors may also be used including sound, flow, optical, and chemical sensors.

Although the techniques for detecting ischemia alerting has been discussed with respect to an implanted system for the detection of cardiac events, it is also envisioned that these techniques are equally applicable to systems for the detection of cardiac events that are entirely external to the patient. For clarity, the time interval between alerting signals within a set (set of what) is hereby termed as the intra-set time interval and the time interval between sets of alerting signals is hereby termed the inter-set time interval.

Various other modifications, adaptations, and alternative designs are of course possible in light of the above teachings. Therefore, it should be understood at this time that, within the scope of the appended claims, the invention can be practiced otherwise than as specifically described herein.

What is claimed is:

1. A method for assessing the condition of the heart of a human patient, the method comprising the steps of:
receiving an electrogram reflecting the electrical activity within the patient's heart,
applying a hierarchical classification scheme to the electrogram based on different features of the electrogram, thereby determining a category for the electrogram, wherein the hierarchical classification scheme comprises a series of classification tests;
estimating the heart's condition based on the category.

2. The method of claim 1 wherein the step of estimating the heart's condition comprises the step of applying a condition test based on the category, wherein the outcome of the condition test is indicative of the heart's condition.

3. The method of claim 1 wherein the step of estimating the heart's condition comprises the step of applying a test based on the category, wherein the outcome of a first one of the classification tests serves as an estimate of the heart's condition.

4. The method of claim 2 wherein the outcome of the condition test is a measure of myocardial ischemia.

5. The method of claim 4 wherein at least one of the classification tests comprises the step of comparing T wave amplitude with a threshold.

6. The method of claim 4 wherein at least one of the classification tests comprises the step of examining the polarity of an ST segment deviation.

7. The method of claim 4 wherein at least one of the classification tests comprises the step of examining the rate of change of a cardiac feature.

8. The method of claim 4 wherein at least one of the classification tests is heart rate dependent.

9. The method of claim 4 wherein the condition test is heart rate dependent.

10. The method of claim 1 wherein the series of classification tests includes at least a first level of classification tests and a second level of classification tests, and wherein at least one result of the first level of classification tests determines at least one test in the second level of classification tests that will be selected to further classify the electrogram.

11. The method of claim 1 wherein the series of classification tests are mutually exclusive.

12. The method of claim 1 wherein the method is realized by an implantable device, the method further comprising alerting the patient based upon estimating the heart's condition.

13. The method of claim 1 wherein the method is realized by an implantable device, the method further comprising providing treatment to the patient based upon estimating the heart's condition.

14. The method of claim 1 wherein estimating the heart's condition based on the category, includes analyzing the electrogram using an algorithm selected based upon the category.

15. A method for detecting a cardiac event, the method comprising the steps of:
a) receiving an electrogram reflecting the electrical activity within the patient's heart,
b) computing a plurality of heart signal feature values from the electrogram;
c) comparing each of a first set of said plurality of heart signal feature values with a corresponding range within a first set of ranges, wherein the values in the first set of ranges are selected to form a combination which is associated with a cardiac event, and wherein the first set of said plurality of heart signal feature values comprises at least two heart signal feature values;
d) comparing each of a second set of said plurality of heart signal feature values with a corresponding range within at a second set of ranges, wherein the values in the second set of ranges are selected to form a combination which is associated with the cardiac event, and wherein the second set of said plurality of heart signal feature values comprises at least two heart signal feature values;
wherein a first one of the ranges in the first set of ranges does not overlap a corresponding range in the second set of ranges, and wherein the first one of the ranges pertains to a heart signal feature other than heart rate, and wherein at least one heart signal feature value in the second set is not within the first set;
e) detecting the cardiac event based on the outcome of steps b and c.

16. The method of claim 15 wherein cardiac event is detected based on the outcome of steps b and c and information from a different electrogram.

17. The method of claim 15 wherein both the first and second sets of ranges include heart signal feature which is the amplitude of the T wave, and the amplitude of the T wave exceeds a threshold in the first set of ranges, heart signal feature and the amplitude of the T wave is less than or equal to the threshold in the second set of heart signal feature ranges (Bruce, this does not seem to make sense).

18. The method of claim 15 wherein steps b and c comprise the steps of accessing a look up table.

19. A method for assessing the condition of the heart of a human patient, the method comprising the steps of:
receiving an electrogram,
applying a classification scheme to the electrogram based on a plurality of features of the electrogram, thereby determining a category for the electrogram, wherein the category is one of a set of non-overlapping categories; and,
estimating the heart's condition based on the category.

20. The method of claim 19, wherein the classification scheme comprises a series of classification tests.

21. The method of claim 19, wherein the category is selected to be one from at least two of the following categories: transmural ischemia; early subendocardial ischemia; late subendocardial ischemia.

22. The method of claim 21, wherein the category is further selected to be one of:
electrogram data from an electrode in an ischemic region; electrogram data from an electrode outside of an ischemic region.

23. The method of claim 21, wherein the category is further selected based upon one of at least two selected heart rate ranges.

24. The method of claim 21, wherein the category is further selected based upon the historical rate of change of at least one feature of the electrogram.

25. The method of claim 21, wherein the category is further selected contingently upon the historical classification of prior electrogram data.

26. The method of claim 21, wherein the category is further selected based upon the history of heart rate data.

27. The method of claim 21, wherein the category is further selected based upon non-cardiac measures of a patient's activity level.

28. A method for detecting a cardiac event, the method comprising the steps of:
a) receiving an electrogram reflecting the electrical activity within the patient's heart,
b) computing a plurality of heart signal feature values from the electrogram;
c) comparing each of a first set of said plurality of heart signal feature values with a corresponding range within a first set of ranges, wherein the values in the first set of ranges are selected to form a combination which is associated with a cardiac event, and wherein the first set of said plurality of heart signal feature values comprises at least two heart signal feature values;
d) comparing each of a second set of said plurality of heart signal feature values with a corresponding range within at a second set of ranges, wherein the values in the second set of ranges are selected to form a combination which is associated with the cardiac event, and wherein the second set of said plurality of heart signal feature values comprises at least two heart signal feature values;
wherein a first one of the ranges in the first set of ranges does not overlap a corresponding range in the second set of ranges, and wherein the first one of the ranges pertains to a heart signal feature other than heart rate, and wherein an increase in a first heart signal feature value in the first set is associated with a cardiac event according to the first set of ranges whereas a decrease in the first heart signal feature value in the second set is associated with a cardiac event according to the second set of ranges;
e) detecting the cardiac event based on the outcome of steps b and c.

29. The method of claim 28 wherein cardiac event is detected based on the outcome of steps b and c and information from a different electrogram.

30. The method of claim 28 wherein both the first and second sets of ranges include the amplitude of the T wave, and the amplitude of the T wave is exceeds a threshold in the first set of ranges, heart signal feature and the amplitude of the T wave is less than or equal to the threshold in the second set of heart signal feature ranges.

31. The method of claim 28 wherein steps b and c comprise the steps of accessing a look up table.
